(19) United States
(12) Patent Application Publication
(10) Pub. No.: US 2026/0130619 A1
(43) Pub. Date: May 14, 2026

(54) NARROW PULSE REJECTION IN ECG MONITORING

(71) Applicant: Draeger Medical Systems, Inc., Andover, MA (US)

(72) Inventors: Yu Chen, Andover, MA (US); Haisheng Lu, Andover, MA (US)

(21) Appl. No.: 19/288,615

(22) Filed: Aug. 1, 2025

Related U.S. Application Data
(60) Provisional application No. 63/679,785, filed on Aug. 6, 2024.

(51) Int. Cl. A61B 5/349; A61B 5/00; A61B 5/308; A61B 5/339

(52) U.S. Cl. CPC A61B 5/349; A61B 5/0006; A61B 5/308; A61B 5/339; A61B 5/7203

(57) ABSTRACT

A computer-implemented method for mitigating noise from QRS complexes extracted from an electrocardiogram (“ECG”) signal includes: pre-processing an acquired ECG signal to obtain a pre-processed ECG signal identifying narrow pulses in the pre-processed ECG signal; extracting QRS complexes from the acquired ECG signal; cross-checking the extracted QRS complexes with the identified narrow pulses; and rejecting pulses in the extracted QRS complexes that synchronize with the identified narrow pulses to obtain denoised QRS complexes. The pre-processing includes applying a first bandpass filter comprised of finite and infinite impulse response filters; and applying a second bandpass filter comprised of an infinite impulse response filter.

### CROSS REFERENCE TO RELATED APPLICATIONS

The present application claims priority to and the benefit of U.S. Prov. Pat. App. Ser. No. 63/679,785, which was filed on Aug. 6, 2024, for all purposes, including the right of priority, which application is hereby incorporated herein by reference in its entirety and to the extent that is not inconsistent with the present disclosure.

### TECHNICAL FIELD

The present disclosure relates generally to the field of electrocardiogram (“ECG”) signal analysis. More particularly, the present disclosure relates to ECG waveform analysis including narrow pulse rejection.

### BACKGROUND

This section of this document introduces information about and/or from the art that may provide context for or be related to the subject matter described herein and/or claimed below. It provides background information to facilitate a better understanding of the various aspects of the present invention. This is a discussion of “related” art. That such art is related in no way implies that it is also “prior” art. The related art may or may not be prior art. The discussion in this section of this document is to be read in this light, and not as admissions of prior art.

Electrocardiogram systems are commonly used to monitor patients'heart conditions as well as to detect or predict cardiac events and conditions. In clinical settings, ECG signals representative of a patient's condition are captured in waveforms and analyzed by physiological monitoring devices. The physiological monitoring devices identify systolic segments of the captured waveforms, such as QRS-complexes, P-Q segments, S-T segments, and the like. These systolic segments reflect the progression of electrical signals in the heart and corresponding to the depolarization of the right and left ventricles and the contraction of cardiac muscles. For a normal sinus rhythm, an R-wave (a sharp upward deflection) in the QRS-complex with a large amplitude and a small width, is suitable for measuring heart rate and other cardiac conditions. Physiological monitoring devices further classify the detected ECG waveforms into different types, based on features extracted from the morphology of various systolic segments.

### SUMMARY

Acquired ECG signals sometimes include narrow pulses that can be mistaken for QRS complexes. It is therefore desirable to detect and reject these narrow pulses before they can be falsely counted as QRS complexes. Furthermore, the time duration for these narrow pulses can vary depending on the age of the patient and there are industry standards that are applicable to ECG analysis that impact this process. (This time duration is sometimes referred to as “width” because of the manner in which acquired ECG signals may be rendered and displayed for human perception.) The presently disclosed technique is directed to narrow pulse detection and rejection in ECG signals prior to QRS complex detection in light of these and other factors.

In a first embodiment, a computer-implemented method for mitigating noise from QRS complexes extracted from an electrocardiogram (“ECG”) signal, comprises: pre-processing an acquired ECG signal to obtain a pre-processed ECG signal. The computer-implemented method includes: pre-processing an acquired ECG signal to obtain a pre-processed ECG signal; identifying narrow pulses in the pre-processed ECG signal; extracting QRS complexes from the acquired ECG signal; cross-checking the extracted QRS complexes with the identified narrow pulses; and rejecting pulses in the extracted QRS complexes that synchronize with the identified narrow pulses to obtain denoised QRS complexes. The pre-processing includes applying a first bandpass filter comprised of finite and infinite impulse response filters; and applying a second bandpass filter comprised of an infinite impulse response filter.

In a second embodiment, a patient monitor, comprises: a sensor interface through which the patient monitor may receive acquired electrocardiogram (“ECG”) signals; one or more processors; and a memory. The memory is encoded with instructions that, when executed by the one or more processors, cause the one or more processors to perform a computer-implemented method for mitigating noise from QRS complexes extracted from an electrocardiogram (“ECG”) signal. The computer-implemented method comprises pre-processing an acquired ECG signal to obtain a pre-processed ECG signal. The computer-implemented method includes: pre-processing an acquired ECG signal to obtain a pre-processed ECG signal; identifying narrow pulses in the pre-processed ECG signal; extracting QRS complexes from the acquired ECG signal; cross-checking the extracted QRS complexes with the identified narrow pulses; and rejecting pulses in the extracted QRS complexes that synchronize with the identified narrow pulses to obtain denoised QRS complexes. The pre-processing includes applying a first bandpass filter comprised of finite and infinite impulse response filters; and applying a second bandpass filter comprised of an infinite impulse response filter.

In a third embodiment, a technological system for delivering care to a patient, the technological system comprising a patient monitor, a network over which the patient monitor transmits signals, and a computing device receiving the signals transmitted by the patient monitor over the network. The patient monitor comprises a sensor interface through which the patient monitor may receive acquired electrocardiogram (“ECG”) signals, a communications interface; a display; one or more processors; and a memory. The computer-implemented method includes: performing a computer-implemented method for mitigating noise from QRS complexes extracted from an electrocardiogram (“ECG”) signal, the computer-implemented method comprising; transmit the acquired ECG signal, or the pre-processed ECG signal, or the extracted QRS complexes, or the denoised QRS complexes, or some combination thereof; and rendering for human perception and displaying the acquired ECG signal, or the pre-processed ECG signal, or the extracted QRS complexes, or the denoised QRS complexes, or some combination thereof.

The computer-implemented method comprises pre-processing an acquired ECG signal to obtain a pre-processed ECG signal. The computer-implemented method includes: pre-processing an acquired ECG signal to obtain a pre-processed ECG signal; identifying narrow pulses in the pre-processed ECG signal; extracting QRS complexes from the acquired ECG signal; cross-checking the extracted QRS complexes with the identified narrow pulses; and rejecting pulses in the extracted QRS complexes that synchronize with the identified narrow pulses to obtain denoised QRS complexes. The pre-processing includes applying a first bandpass filter comprised of finite and infinite impulse response filters; and applying a second bandpass filter comprised of an infinite impulse response filter.

The above presents a simplified summary in order to provide a basic understanding of some aspects of what is claimed below. This summary is not an exhaustive overview of the claimed subject matter. It is not intended to identify key or critical elements of the disclosure or to delineate the scope of the claims. Its sole purpose is to present some concepts in a simplified form as a prelude to the more detailed description that is discussed below.

### BRIEF DESCRIPTION OF THE DRAWINGS

In the drawings, like reference numbers generally indicate identical, functionally similar, and/or structurally similar elements.

FIG. 1 illustrates a patient monitoring system according to one or more examples.

FIG. 2 is a schematic representation of a “normal” sinus rhythm ECG wave illustrating selected characteristics thereof;

FIG. 3 illustrates a computer-implemented method for mitigating noise from QRS complexes extracted from an electrocardiogram (“ECG”) signal in accordance with one or more embodiments.

FIG. 4 illustrates one particular implementation for the pre-processing of the acquired ECG signal to obtain a pre-processed ECG signal in the computer-implemented method of FIG. 3.

FIG. 5 illustrates one particular implementation for the narrow pulse identification in the pre-processed ECG signal in the computer-implemented method of FIG. 3.

FIG. 6 illustrates one particular implementation for the QRS complex extraction in the computer-implemented method of FIG. 3.

FIG. 7 illustrates one particular scenario in which acquired data is both processed, analyzed, and used in both local and remote locations in accordance with one of more embodiments.

FIG. 8 illustrates one particular implementation of the narrow pulse rejection technique disclosed herein.

FIG. 9 graphs a detected peak.

FIG. 10 illustrates certain aspects of the analysis.

FIG. 11 graphs a detected peak over time that will be rejected as a narrow pulse in a QRS complex.

While the disclosed subject matter is susceptible to various modifications and alternative forms, the drawings illustrate specific implementations described in detail by way of example. It should be understood, however, that the description herein of specific examples is not intended to limit that which is claimed to the particular forms disclosed, but on the contrary, the intention is to cover all modifications, equivalents, and alternatives falling within the spirit and scope of the appended claims.

### DETAILED DESCRIPTION

Illustrative examples of the subject matter claimed below are disclosed. In the interest of clarity, not all features of an actual implementation are described for every example in this specification. It will be appreciated that in the development of any such actual implementation, numerous implementation-specific decisions may be made to achieve the developers'specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

As noted above, there are several challenges in narrow pulse rejection in QRS complex detection of ECG signals. One noted challenge is that QRS complex width can vary depending on the age of the patient. For example, QRS complexes in children may be as low as 20-90 ms for a “normal” ECG, including both neonatal and pediatric patients. QRS complexes in adults, on the other hand, may have a width of 60-120 ms for a “normal” ECG. A second challenge arises from industry standards. For instance, the Association for the Advancement of Medical Instrumentation® (“AAMI”) 60601-2-27 standard requires that the heart rate meter shall not respond to ECG signals having a QRS duration of 10 ms or less with an amplitude of 1 mV. The technical challenge is the 10 ms QRS pulse could be smeared out by the low pass filter of the monitoring ECG signal band 0.5-40 Hz. To pass the AAMI 60601-2-27 standard testing, the high QRS detection threshold 0.4 mV is used, contradicting to the clinical needs for low threshold to better detect small QRS complexes.

The presently disclosed technique provides a low QRS complex detection threshold 0.18-0.2 mV to meet clinical needs (20 ms and above) while rejecting 10 ms pulses in the full amplitude range. A 0.44 mV, 10 ms pulse is at the predefined threshold 41 uV in narrow pulse analysis. However, its amplitude in filtered 0.5-40 Hz is only 0.175 mV, below the QRS 0.18-0.2 mV detection threshold. Any 10 ms pulse with amplitude greater than 0.44 mV is above the predefined threshold 40μV in narrow pulse analysis. A cross check would reject the QRS complex detected in 0.5-40 Hz.

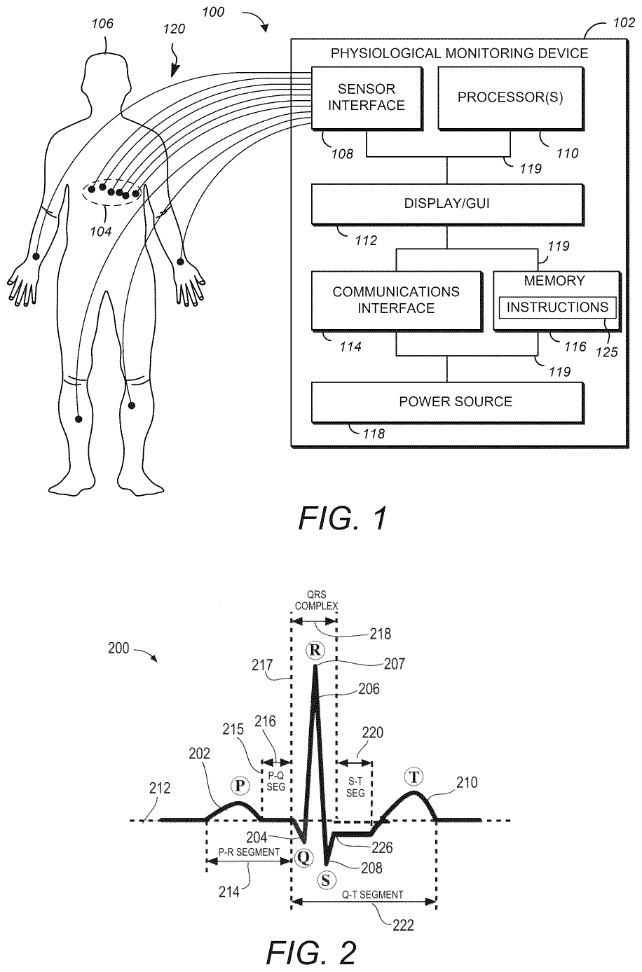

Turning now to the drawings, FIG. 1 illustrates a physiological monitoring system 100 according to one or more examples. As shown in FIG. 1, the system 100 includes a patient monitor 102 capable of receiving physiological data from various sensors 104 connected to a patient 106 when deployed. In this example, the plurality of sensors 104 comprise electrocardiogram (“ECG”) electrodes affixed to the skin of patient 106.

In general, it is contemplated by the present disclosure that patient monitor 102 includes electronic components and/or electronic computing devices operable to receive, transmit, process, store, and/or manage patient data and information associated performing the functions of the system as described herein, which encompasses any suitable processing device adapted to perform computing tasks consistent with the execution of computer-readable instructions stored in a memory or a computer-readable recording medium.

Further, any, all, or some of the computing devices in patient monitor 102 may be adapted to execute any operating system, including Linux®, UNIX®, Windows Server®, etc., as well as virtual machines adapted to virtualize execution of a particular operating system, including customized and proprietary operating systems. Patient monitor 102 may be further equipped with components to facilitate communication with other computing devices over one or more network connections, which may include connections to local and wide area networks, wireless and wired networks, public and private networks, and any other communication network enabling communication in the system.

As shown in FIG. 1, patient monitor 102 may be, for example, a patient monitor implemented to monitor various physiological parameters of patient 106 via sensors 104. Patient monitor 102 may include a sensor interface 108, one or more processors 110, a display/graphical user interface (“GUI”) 112, a communications interface 114, a memory 116, and a power source (or power connection) 118, all communicating over an internal bus 119. The sensor interface 108 may be implemented in hardware or combination of hardware and software and is used to connect via wired and/or wireless connections to the sensors 104 for gathering physiological data from the patient 106. As noted, the sensors 104 in the present example are ECG electrodes affixed to the skin of the patient 106. A plurality of conductive leads 120, comprising a plurality of conductive cables, are provided for coupling the sensors 104 to the sensor interface 108. In one or more examples, the conductive leads 120 comprise a plurality of ECG cables.

The data signals from the sensors 104 may include, for example, sensor data related to an ECG. The one or more processors 110 may be used for controlling the general operations of the patient monitor 102, as well as processing sensor data received by sensor interface 108. The one or more processors 110 may be any suitable processor-based resource. They may be, but are not limited to, a central processing unit (“CPU”), a hardware microprocessor, a multi-core processor, a single core processor, a field programmable gate array (“FPGA”), a controller, a microcontroller, an application specific integrated circuit (“ASIC”), a digital signal processor (“DSP”), or other similar processing device capable of executing any type of instructions, algorithms, or software for controlling the operation and performing the functions of patient monitor 102. In some embodiments, the one or more processors 110 may comprise a processor chipset including, for example and without limitation, one or more co-processors.

The display/GUI 112 may be configured to display various patient data, sensor data, and hospital or patient care information, and includes a user interface implemented for allowing interaction and communication between a user and patient monitor 102. The display/GUI 112 may include a keyboard (not shown) and/or pointing or tracking device (not shown), as well as a display, such as a liquid crystal display (“LCD”), cathode ray tube (“CRT”) display, thin film transistor (“TFT”) display, light-emitting diode (“LED”) display, high definition (“HD”) display, or other similar display device that may include touch screen capabilities. The display/GUI 112 may provide a means for inputting instructions or information directly to the patient monitor 102. The patient information displayed may, for example, relate to the measured physiological parameters of the patient 106 (e.g., ECG readings).

The communications interface 114 may permit the patient monitor 102 to directly or indirectly (via, for example, a monitor mount) communicate with one or more computing networks and devices, workstations, consoles, computers, monitoring equipment, alert systems, and/or mobile devices (e.g., a mobile phone, tablet, or other hand-held display device). The communications interface 114 may include various network cards, interfaces, communication channels, cloud, antennas, and/or circuitry to permit wired and wireless communications with such computing networks and devices. The communications interface 114 may be used to implement, for example, a BLUETOOTH® connection, a cellular network connection, and/or a WIFI® connection with such computing networks and devices. Example wireless communication connections implemented using the communication interface 114 include wireless connections that operate in accordance with, but are not limited to, IEEE802.11 protocol, a Radio Frequency For Consumer Electronics (“RF4CE”) protocol, and/or IEEE802.15.4 protocol (e.g., ZigBee® protocol). In essence, any wireless communication protocol may be used.

Additionally, the communications interface 114 may permit direct (i.e., device-to-device) communications (e.g., messaging, signal exchange, etc.) such as from a monitor mount to patient monitor 102 using, for example, a universal serial bus (“USB”) connection or other communication protocol interface. The communication interface 114 may also permit direct device-to-device connection to other devices such as to a tablet, computer, or similar electronic device; or to an external storage device or memory.

The memory 116 may be a single memory device or one or more memory devices at one or more memory locations that may include, without limitation, one or more of a random-access memory (“RAM”), a memory buffer, a hard drive, a database, an erasable programmable read only memory (“EPROM”), an electrically erasable programmable read only memory (“EEPROM”), a read only memory (“ROM”), a flash memory, hard disk, various layers of memory hierarchy, or any other non-transitory computer readable medium. The memory 116 may be on-chip or off-chip depending on the implementation of the one or more processors 110. The memory 116 may be used to store any type of instructions 125 and patient data associated with algorithms, processes, or operations for controlling the general functions and operations of the patient monitor 102.

The power source 118 may include a self-contained power source such as a battery pack and/or include an interface to be powered through an electrical outlet, either directly or by way of a monitor mount. The power source 118 may also be a rechargeable battery that can be detached allowing for replacement. In the case of a rechargeable battery, a small built-in back-up battery (or super capacitor) can be provided for continuous power to be provided to the patient monitor 102 during battery replacement. Communication between the components of the patient monitor 102 in this example (may be established using the internal bus 119.

The patient monitor 102 may be attached to one or more of several different types of sensors 104 and may be configured to measure and readout physiological data related to patient 106. As noted, the sensors 104 may be attached to the patient monitor 102 by the conductive leads 120 which may be, for example, cables coupled to sensor interface 108. Additionally, or alternatively, one or more sensors 104 may be connected to sensor interface 108 via a wireless connection. In which case sensor interface 108 may include circuity for receiving data from and sending data to one or more devices using, for example, a WIFI® connection, a cellular network connection, and/or a BLUETOOTH® connection.

The data signals received from the sensors 104 may be analog signals. For example, the data signals for the ECG may be input to the sensor interface 108, which can include an ECG data acquisition circuit (not shown separately in FIG. 1). An ECG data acquisition circuit may include amplifying and filtering circuity as well as analog-to-digital (A/D) circuity that converts the analog signal to a digital signal using amplification, filtering, and A/D conversion methods. In the event that the ECG sensor is a wireless sensor, the sensor interface 108 may receive the data signals from a wireless communication module (not shown in FIG. 1). Thus, the sensor interface 108 is a component which may be configured to interface with the one or more sensors 104 and receive sensor data therefrom.

As further described herein, the processing performed by an ECG data acquisition circuit may generate analog data waveforms or digital data waveforms that are analyzed by, in this particular embodiment, a microcontroller. However, other embodiments may use other kinds of processors disclosed above. The microcontroller may be one of the processors 110.

The one or more processors 110, for example, may analyze the ECG waveforms to identify certain waveform characteristics and threshold levels indicative of conditions (abnormal and normal) of the patient 106 using one or more monitoring methods. A monitoring method may include comparing an analog or a digital waveform characteristic or an analog or digital value to one or more threshold values and generating a comparison result based thereon. The microcontroller may be, for example, a processor, an FPGA, an ASIC, a DSP, a microcontroller, or similar processing device.

The microcontroller may include a memory (an on-chip memory) or use a separate memory 116 (an off-chip memory). The memory may be, for example, a RAM, a memory buffer, a hard drive, a database, an EPROM, an EEPROM, a ROM, a flash memory, a hard disk, or any other non-transitory computer readable medium. The memory 116 may store software or algorithms with executable instructions and the microcontroller may execute a set of instructions of the software or algorithms in association with executing different operations and functions of the patient monitor 102 such as analyzing the digital data waveforms related to the data signals from the sensors 104.

To further an understanding of the techniques disclosed herein, a short discussion of QRS complexes will now be presented. FIG. 2 is a schematic representation of a “normal” sinus rhythm ECG wave 200. illustrates a sensed ECG signal corresponding to various phases of a single cardiac beat. As shown in FIG. 2, ECG wave 200 includes a plurality of distinct phases corresponding to periods of polarization and depolarization of regions of the cardiac muscle. In particular, ECG wave 200 includes a P-wave 202, a Q-wave 204, an R-wave 206 having a peak at a fiducial point 207 (also referred to herein as an “R-point”), an S-wave 208, and a T-wave 210. Each of these waves represents either a positive or negative polarization relative to an ECG baseline 212.

A normal sinus rhythm ECG wave such as ECG wave 200 is commonly characterized according to a number of segments, including, as shown in FIG. 2, a P-R segment 214, a P-Q segment 216, a QRS complex 218, an S-T segment 220, and a Q-T segment 222. A normal sinus rhythm ECG wave such as ECG wave 200 may further be characterized by an “isoelectric point” (occurring at dashed line 215 in FIG. 2), an “onset” (occurring at dashed line 217 in FIG. 2), and a junction point or “J-point” 226. The J-point is the junction between the termination of the QRS complex 218 and the onset of the S-T segment 220. Referring to FIG. 2, J-point 226 in ECG wave 200 is the point where QRS complex 218 joins the S-T segment 220. J-point 226 represents the approximate end of depolarization and the beginning of repolarization. J-point 226 may deviate from baseline 212.

As noted above, noise and artifacts may be introduced into sensed ECG signals, such as baseline wander caused by motion of the patient or the leads, muscle/electromyographc (“EMG”) artifacts, spectrum overlapping with the ECG signal, electrode motion artifacts, respiration artifacts, lead placement artifacts, and so on, which can cause the baseline (such as baseline 212 in FIG. 2) to vary. Variability of the ECG baseline can make it difficult to clinically assess a patient's ECG reading and reliably identify the various waves and segments in the sensed ECG waveform.

Referring again to FIG. 1, the one or more processors 110 may further execute under programmed control processes for performing systolic segment measurements, such as S-T segment measurements, Q-T segment measurements, QRS-complex detection, and QRS-complex feature extraction. A QRS-complex is commonly the central and most visually obvious part of an ECG waveform, with a duration of approximately 80 milliseconds (“msec”)-100 msec in adults. Patient monitor 102 may identify the QRS-complex by, for example, identifying an R-wave within the QRS-complex. Processor(s) 110 may search one or more edge points of received sample signals, including a starting point, a peak point, a tail point, and an endpoint. By defining one or more edge points, processor(s) 110 may identify the R-wave and its corresponding QRS-complex.

Concurrently or subsequently, patient monitor 102 may further extract one or more features from the identified QRS-complexes, including but not limited to amplitude, width, morphology, curvature, symmetry, peak direction, segments of different waves including R-R segments (i.e., the time interval between two consecutive R-waves), P-R segments (time interval between the beginning of the upslope of the P wave to the beginning of QRS wave), S-T segment measurements, and Q-T segment measurements. Based on these extracted features, patient monitor 102 may further classify the QRS-complexes into different types referred to as “beats”, including a normal beat or a bundle branch block beat (“N”), a ventricular ectopic beat (“V”), a supraventricular ectopic beat (“S”), a fusion of ventricular and normal beat (“” F) and a paced beat or a beat that cannot be classified (“Q”). Each beat type has its characteristic features and accordingly, patient monitor 102 may store ECG template databases including various pre-determined threshold values or ranges of pre-determined threshold values for each feature. When a new QRS-complex is identified, patient monitor may extract one or more features and compare them with pre-determined threshold values or a ranges of threshold values, thereby classifying the QRS-complex into a specific beat type based on the comparison results.

The Q-T segment of an ECG waveform represents the duration of ventricular depolarization and subsequent repolarization, i.e., from the onset point though the end of the T-wave. The Q-T segment may be used clinically as an indirect measure of the repolarization time. Acute increases in the Q-T segment may be observed in multiple clinical situations and may be associated with an increased risk of syncope and sudden death from ventricular arrhythmias. The Q-T segment may be monitored periodically for possible prolongation. Thus, in some situations, it may be desirable to accurately and continuously monitor the Q-T segment in real time in a reliable manner, and to accurately detect feature points of the onset point and the T-wave end in ECG signals.

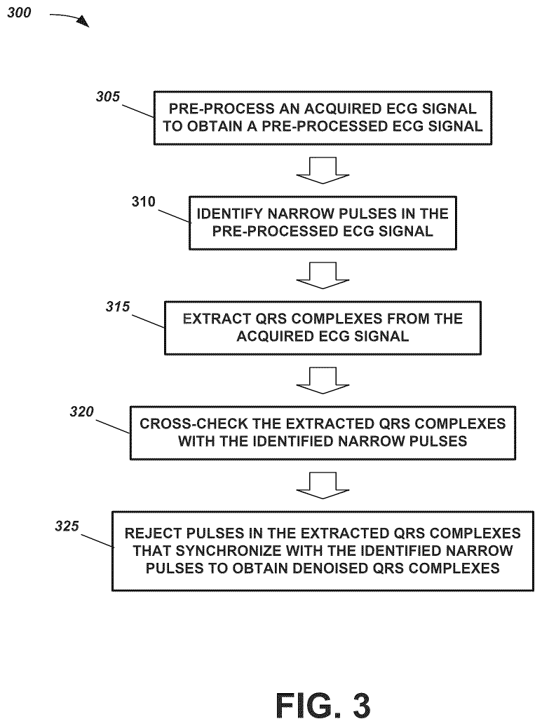

FIG. 3 illustrates a computer-implemented method 300 for mitigating noise from QRS complexes, such as the QRS complex 200 in FIG. 2, extracted from an electrocardiogram (“ECG”) signal in accordance with one or more embodiments. The method 300 may be performed by the one or more processors 110, shown in FIG. 1, as programmed by instructions 125 stored in the memory 116 as mentioned above. The method 300 presumes that the ECG signal from which the QRS complexes are extracted has been previously acquired using conventional techniques.

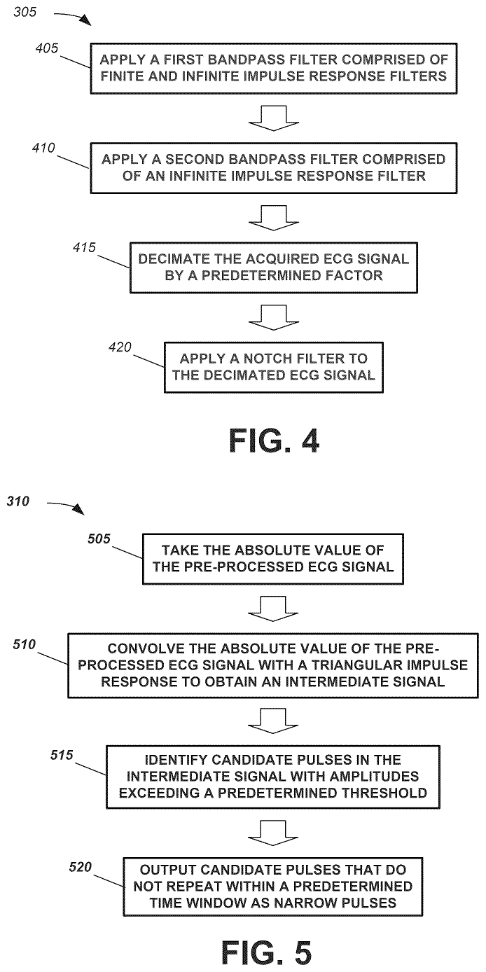

Thus, the method 300 is performed upon an “acquired ECG signal”. The method 300 begins by pre-processing (at 305) an acquired ECG signal to obtain a pre-processed ECG signal. The pre-processing (at 305) may include, as shown in FIG. 4, applying (at 405) a first bandpass filter comprised of finite and infinite impulse response filters and applying (at 410) a second bandpass filter comprised of an infinite impulse response filter. In some embodiments, the pre-processing (at 305) may optionally further include decimating (at 415) the acquired ECG signal by a predetermined factor and applying (at 420) a notch filter to the decimated ECG signal.

Returning to FIG. 3, the method 300 then identifies (at 310) narrow pulses in the pre-processed ECG signal. The identification (at 310) may include, as shown in FIG. 5, taking (at 505) the absolute value of the pre-processed ECG signal and then convolving (at 510) the absolute value of the pre-processed ECG signal with a triangular impulse response to obtain an intermediate signal. Next, candidate pulses are identified (at 515) in the intermediate signal with amplitudes exceeding a predetermined threshold. Candidate pulses that do not repeat within a predetermined time window are then output (at 520) as narrow pulses.

In one embodiment, a “narrow” pulse is characterized as a QRS width is less than 15 ms although this may vary depending upon the embodiment.

Also, in one particular embodiment, the “predetermined time window” is 200 ms, although, again, this may vary depending upon the embodiment.

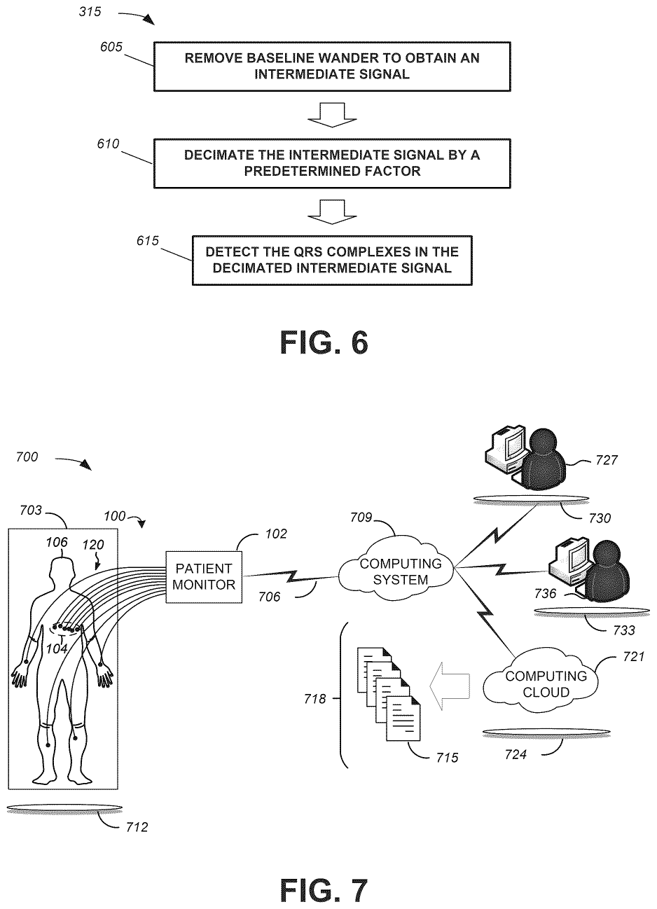

Returning again to FIG. 3, the method 300 also extracts (at 315) QRS complexes from the acquired ECG signal. As shown in FIG. 6, extracting (at 315) the QRS complexes may include removing (at 605) baseline wander to obtain an intermediate signal. The intermediate signal is then decimated (at 610) by a predetermined factor and the QRS complexes are detected (at 615) in the decimated intermediate signal.

It will be appreciated that the order in which the various actions of the method 300 are presented in FIG. 3 or in this disclosure does not necessarily impose any kind of ordering on the performance of those actions. In particular, the pre-processing (at 305) and narrow pulse identification (at 310) need not necessarily be performed prior to the QRS complex extraction (at 315). Indeed, the QRS complex extraction (at 315) may be performed prior to or in parallel with the pre-processing (at 305) and narrow pulse identification (at 310).

Once the narrow pulses are identified and the QRS complexes are identified, the method 300 then cross-checks (at 320) the extracted QRS complexes with the identified narrow pulses. The identified narrow pulses that synchronize with the extracted QRS complexes are presumed to be noise, and therefore undesirable. The method 300 therefore then rejects (at 325) pulses in the extracted QRS complexes that synchronize with the identified narrow pulses to obtain denoised QRS complexes.

The denoised QRS complexes may find many uses in a clinical setting for monitoring, evaluating, and treating a patient from whom they are obtained. Accordingly, in some embodiments, the method 300 in FIG. 3 may be performed on board the patient monitor, e.g., the patient monitor 102 in FIG. 1, whereupon the patient monitor may transmit one or more of the acquired ECG signal, or the pre-processed ECG signal, or the extracted QRS complexes, or the denoised QRS complexes. For example, in an embodiment in which the method 300 is implemented on the patient monitor 102, the one or more processors 110 may transmit the acquired ECG signal, or the pre-processed ECG signal, or the extracted QRS complexes, or the denoised QRS complexes, or some combination thereof off monitor via the communications interface 114. One such embodiment is discussed below.

The signals may also find ample use on the patient monitor, typically at a patient's bedside. In embodiments in which the patient monitor includes a display, e.g., the display/GUI 112 of the patient monitor 102 in FIG. 1, one or more of the signals may be rendered for human perception and displayed. Thus, the patient monitor may render for human perception and display the acquired ECG signal, or the pre-processed ECG signal, or the extracted QRS complexes, or the denoised QRS complexes, or some combination thereof. Again, one such embodiment is disclosed below.

The method(s)s of the techniques disclosed herein, as well as variations thereon disclosed herein, may be performed in either hardware, software, or a combination thereof. In the illustrated embodiments, the method(s) are performed in software only. More particularly, and returning to FIG. 1, the method(s) may be executed by the one or more processors 110 as programmed by the instructions 125 residing on the memory 116. In this particular embodiment, the one or more processors 110 is a single microprocessor and the memory 116 is a local, random-access memory (“RAM”).

However, as discussed above, the claimed subject matter admits wide variation in this aspect. For example, the one or more processors 110 and memory 116 may be implemented in a “System on a Chip” (“SoC”) with the sensor interface 108, and the communications interface 114. For a second example, the one or more processors 110 and memory 116 may be implemented in an appropriately programmed Electrically Programmable Read Only Memory (“EPROM”) or some similar device that is both processor and memory. For a third example, the one or more processors 110 and the memory 116 may be implemented in an Application Specific Integrated Circuit (“ASIC”). Those skilled in the art having the benefit of this disclosure may appreciate still other variations on this theme.

Data may be acquired and processed on the patient monitor, or data may be transmitted from the patient monitor off monitor to a remote location for use and/or analysis, or some combination thereof. As used herein and in this context, “remote” means outside the physical presence of the patient monitor. Conversely, “local” means in the physical presence of the patient monitor. Such remote locations may include, without limitation, a central monitoring or nurses'station outside the patient's room, or in physician's office in another facility, or in a computing cloud located in another facility. Those skilled in the art having the benefit of this disclosure may appreciate still other variations on this theme.

FIG. 7 illustrates one particular scenario in which acquired data is both processed, analyzed, and used in both local and remote locations in accordance with one of more embodiments. In the scenario 700, a physiological monitoring system 100, first shown in FIG. 1, includes a patient monitor 102 and a plurality of conductive leads 120 as described above. The conductive leads 120 are affixed to the patient 106 disposed upon a bed or pallet 703 and data is being acquired.

In this embodiment, the data is processed by the patient monitor 102 using the one or more processors 110 programmed to execute the instructions 125, both shown in FIG. 1, using the method 300, shown in FIG. 3-FIG. 6. The results are then rendered for human perception and displayed on the display/GUI 112. This permits a caregiver, not shown, to monitor the condition of the patient 106 at the bedside. Note that the patient monitor 102 may also display a variety of other information pertaining to the care and condition of the patient 106.

The scenario 700 then contemplates that the patient monitor 102 will transfer the raw, acquired data and/or the processed results from the local location 712 off monitor using the communications interface 114, shown in FIG. 1, over the communications link 706 and the computing system 709. The communications link 706 may be wired, wireless, or some combination, depending on the implementation. Similarly, the computing system 709 may include, without limitation, a private network, such as a local area network in the facility in which the patient monitor 102 is located, a public network, such as the Internet, or some combination of private and public networks.

In some embodiments, including the scenario 700, the raw, acquired data and/or processed results may be stored in an electronic medical record (“EMR”) 715 that is a part of an EMR repository 718 residing in a computing cloud 721 at a remote location 724. From there, a physician or other caregiver 727 at a remote location 730 may access the EMR 715 for monitoring or analysis. The remote location 730 may be, for example, an office in the facility in which the patient 102 is admitted or in a separate facility in which the physician 727 maintains an office.

The scenario 700 also contemplates that the transmitted data and/or processed results may be received and displayed in real time or near real time at remote locations such as the remote locations 730, 733. As used herein in this context, the term “near real time” means as close to real time as available computing resources will permit. For instance, the remote location 733 may be a nurses'station or centralized monitoring station located in the same facility in which the patient 106 is admitted, where a caregiver 736 may monitor the condition of the patient 106. As mentioned immediately above, the remote location 730 may be, for example, an office in the facility in which the patient 102 is admitted or in a separate facility in which the physician 727 maintains an office.

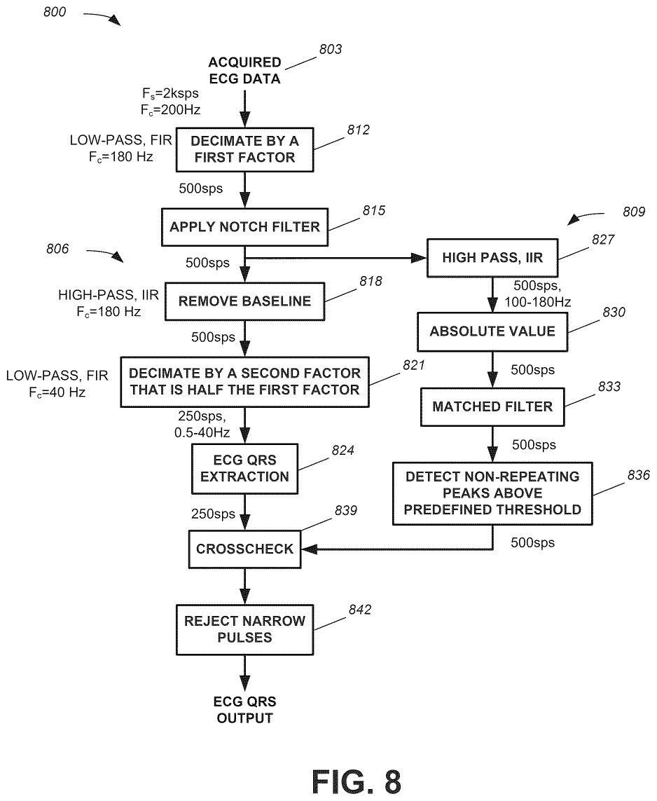

FIG. 8 illustrates one particular implementation of the narrow pulse rejection technique disclosed herein. The implementation 800, like the method 300 in FIG. 3, assumes prior acquisition (at 803) of ECG data. Note also the parallel paths 806, 809 of execution in the implementation 800. As was discussed above, the execution of the steps in the method 300 may be linear or non-linear and their ordering as presented in FIG. 3 is not controlling. One consequence of these observations is that parallel execution as shown in the implementation 800 in FIG. 8 is contemplated in some embodiments.

The implementation 800 receives the acquired ECG data at a sampling rate Fₛ=2 ksps and a cutoff filter frequency F_{c}=200 Hz. The received ECG data is then decimated by a first factor (at 812). T/his includes applying a low-pass, finite impulse response (“FIR”) filter with F_{c}=180 Hz. In the implementation, the factor is four to Fₛ=500 sps. A notch filter is then applied (at 815). Execution then splits into the parallel paths 806, 809 as discussed above. The parallel path 806 begins by removing the baseline (at 818). In this particular embodiment this includes applying a high-pass, infinite impulse response (“IIR”) with F_{c}=180 Hz. The data is then decimated by a second factor that is half the first factor (at 821). The ECG QRS complexes are then extracted (at 824).

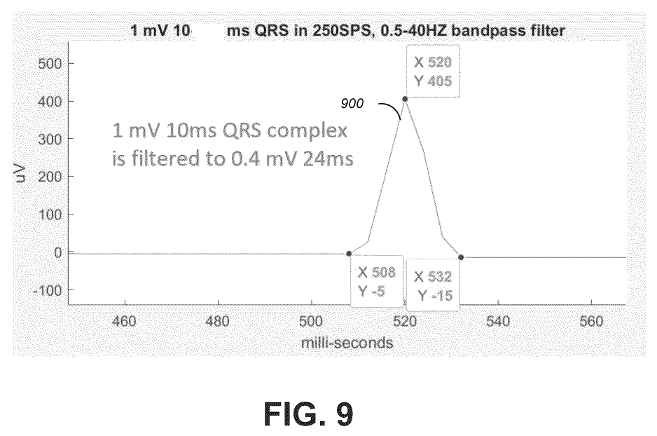

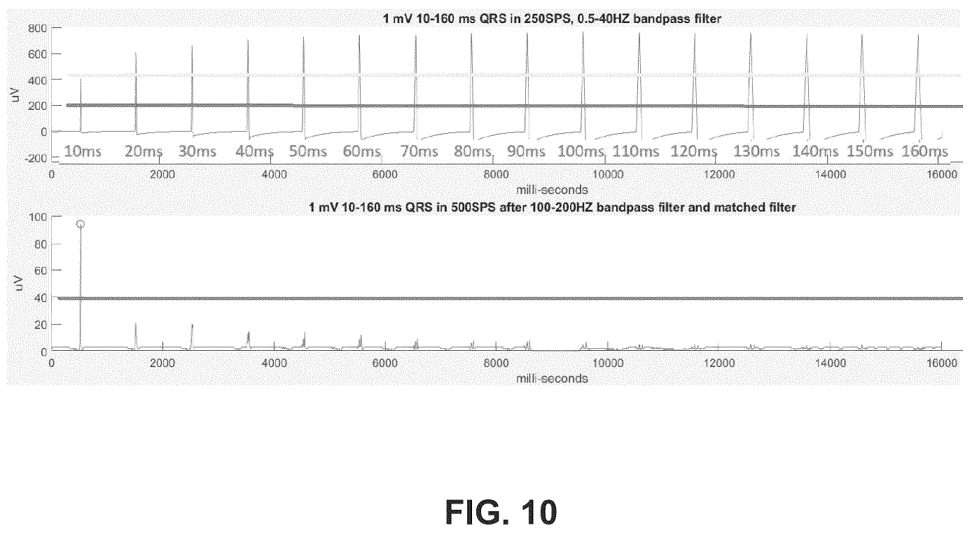

Meanwhile, in the parallel path 809, a high-pass IIR filter is applied (at 827) to the data for Fₛ=500 sps and F_{c}=100-180 Hz. The absolute value is taken (at 830) and a matched filter applied (at 833). In the implementation 800, this includes filtering the data by convolving it with a triangular impulse response. This approximates a matched filter related to the QRS complex and provides a stable fiducial point determination over other low pass filters. Non-repeating peaks above a predefined threshold are then detected (at 836). In the implementation 800, “non-repeating” means occurring no more than once in a 200 ms window. More particularly, the implementation 800 identifies the peaks in the data over the predefined threshold (40 μV, red line in FIG. 10, lower plot), and outputs the peaks only detected once in 200 ms window. One such peak 900 is illustrated in FIG. 9.

The parallel paths 806, 809 then rejoin at the crosscheck (at 839) between the extracted QRS complexes (from 824) and the detected peaks (i.e., narrow pulses, from 836). More particularly, the implementation 800 cross checks the QRS complexes detected in 0.5-40 Hz bandwidth and rejects the pulses synchronizing with the output from narrow pulse analysis (i.e., the parallel path 809). The narrow pulses are then rejected (at 842).

FIG. 10 illustrates certain aspects of the analysis discussed above. FIG. 10 shows, in the top graph, a 10 ms 1 mV narrow pulse in 250 SPS signal is detected as QRS at desired threshold 0.18-0.2 mV (red line), but below 0.4 mV high threshold (yellow line). It also demonstrates in the lower graph, a 10 ms 1 mV narrow pulse in 500 SPS signal that is recognized as noise by threshold 40 (red line)

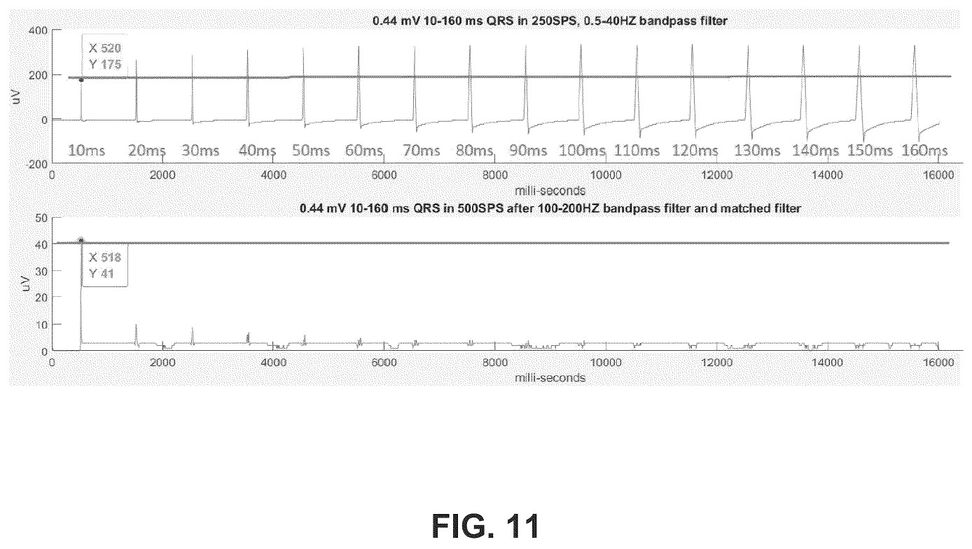

The result in a denoised QRS output comprised of QRS complexes from which narrow pulses have been removed, or at least mitigated. A low QRS detection threshold 0.18-0.2 mV can be achieved to meet clinical needs (20 ms and above) and reject 10 ms pulses in the full amplitude range. FIG. 11 graphs a detected peak that will be rejected as a narrow pulse in a QRS complex. As illustrated in FIGS. 11, 0.44 mV 10 ms pulse is at the predefined threshold 41 μV in narrow pulse analysis (lower plot), but its amplitude in filtered 0.5-40 Hz is only 0.175 mV (upper plot), below the QRS 0.18-0.2 mV detection threshold. Any 10 ms pulse with amplitude greater than 0.44 mV is above the predefined threshold 40 μV in narrow pulse analysis. The cross check would reject the QRS complex detected in 0.5-40 Hz.

In various examples, hardware processor(s) 110 in monitoring device 102 may be, for example and without limitation, a microcontroller, a central processing unit (“CPU”), a digital signal processor (“DSP”), a programmed logic array (“PLA”), or a custom processing circuit. Instructions may be executed by one or more processors, such as one or more central processing units (“CPU”), digital signal processors (“DSPs)”, general purpose microprocessors, application specific integrated circuits (“ASICs”), field programmable logic arrays (“FPGAs”), or other equivalent integrated or discrete logic circuitry. Accordingly, the term “processor,” as used herein refers to any of the foregoing structure or any other structure suitable for implementation of the techniques described herein. In addition, in some aspects, the functionality described herein may be provided within dedicated hardware and/or software modules. Also, the techniques could be fully implemented in one or more circuits or logic elements. A “controller,” including one or more processors, may use electrical signals and digital algorithms to perform its receptive, analytic, and control functions, which may further include corrective functions. Thus, a controller is a specific type of processing circuitry, comprising one or more processors and memory, that implements control functions by way of generating control signals.

A computer-readable media may be any available media that may be accessed by a computer. By way of example, such computer-readable media may comprise random access memory (“RAM”), read-only memory (“ROM”), electrically-erasable/programmable read-only memory (“EEPROM”), compact disc ROM (“CD-ROM”) or other optical disk storage, magnetic disk storage or other magnetic storage devices, or any other medium that may be used to carry or store desired program code in the form of instructions or data structures and that may be accessed by a computer. Disk and disc, as used herein, includes compact disc (“CD”), laser disc, optical disc, digital versatile disc (“DVD”), floppy disk and Blu-ray® disc where disks usually reproduce data magnetically, while discs reproduce data optically with lasers.

Note also that the software implemented aspects of the subject matter hereof are usually encoded on some form of program storage medium or implemented over some type of transmission medium. The program storage medium is a non-transitory medium and may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The claimed subject matter is not limited by these aspects of any given implementation.

Accordingly, in a first embodiment, a computer-implemented method for mitigating noise from QRS complexes extracted from an electrocardiogram (“ECG”) signal, comprisines: pre-processing an acquired ECG signal to obtain a pre-processed ECG signal, including: identifying narrow pulses in the pre-processed ECG signal; extracting QRS complexes from the acquired ECG signal; cross-checking the extracted QRS complexes with the identified narrow pulses; and rejecting pulses in the extracted QRS complexes that synchronize with the identified narrow pulses to obtain denoised QRS complexes. The pre-processing may include: applying a first bandpass filter comprised of finite and infinite impulse response filters; and applying a second bandpass filter comprised of an infinite impulse response filter.

In a second embodiment, in the computer-implemented method of the first embodiment, identifying narrow pulses in the pre-processed ECG signal may include: taking the absolute value of the pre-processed ECG signal; convolving the absolute value of the pre-processed ECG signal with a triangular impulse response to obtain an intermediate signal; identifying candidate pulses in the intermediate signal with amplitudes exceeding a predetermined threshold; and outputting candidate pulses that do not repeat within a predetermined time window as narrow pulses.

In a third embodiment, in the computer-implemented method of the first embodiment, identifying narrow pulses in the pre-processed ECG signal is performed in parallel with extracting QRS complexes from the acquired ECG signal.

In a fourth embodiment, in computer-implemented method of the first embodiment, pre-processing the acquired ECG signal may further include: decimating the acquired ECG signal by a predetermined factor; and applying a notch filter to the decimated ECG signal.

In a fifth embodiment, in the computer-implemented method of the first embodiment, extracting the QRS complexes from the pre-processed ECG signal may include: removing baseline wander to obtain an intermediate signal; decimating the intermediate signal by a predetermined factor; and detecting the QRS complexes in the decimated intermediate signal.

In a sixth embodiment, a patient monitor, comprises: sensor interface, one or more processors, and a memory. The patient monitor may receive acquired electrocardiogram (“ECG”) signals through the sensor interface. The memory is encoded with instructions that, when executed by the one or more processors, cause the one or more processors to perform a computer-implemented method for mitigating noise from QRS complexes extracted from an electrocardiogram (“ECG”) signal. The computer-implemented method may comprise: pre-processing an acquired ECG signal to obtain a pre-processed ECG signal, including: identifying narrow pulses in the pre-processed ECG signal; extracting QRS complexes from the acquired ECG signal; cross-checking the extracted QRS complexes with the identified narrow pulses; and rejecting pulses in the extracted QRS complexes that synchronize with the identified narrow pulses to obtain denoised QRS complexes. The pre-processing may include: applying a first bandpass filter comprised of finite and infinite impulse response filters; and applying a second bandpass filter comprised of an infinite impulse response filter.

In a seventh embodiment, the patient monitor of the sixth embodiment may identify narrow pulses in the pre-processed ECG signal by taking the absolute value of the pre-processed ECG signal; convolving the absolute value of the pre-processed ECG signal with a triangular impulse response to obtain an intermediate signal; identifying candidate pulses in the intermediate signal with amplitudes exceeding a predetermined threshold; and outputting candidate pulses that do not repeat within a predetermined time window as narrow pulses.

In an eighth embodiment, in the patient monitor of the sixth embodiment, identifying narrow pulses in the pre-processed ECG signal may be performed in parallel with extracting QRS complexes from the acquired ECG signal.

In a ninth embodiment, in the patient monitor of the sixth embodiment, pre-processing the acquired ECG signal further includes: decimating the acquired ECG signal by a predetermined factor; and applying a notch filter to the decimated ECG signal.

In a tenth embodiment, in the patient monitor of the sixth embodiment, extracting the QRS complexes from the pre-processed ECG signal includes: removing baseline wander to obtain an intermediate signal; decimating the intermediate signal by a predetermined factor; and detecting the QRS complexes in the decimated intermediate signal.

In an eleventh embodiment, the patient monitor of the sixth embodiment further comprises a communications interface. The instructions, when executed by the one or more processors, further cause the one or more processors to transmit the acquired ECG signal, or the pre-processed ECG signal, or the extracted QRS complexes, or the denoised QRS complexes, or some combination thereof.

In a twelfth embodiment, the patient monitor of the sixth embodiment further comprises a display. The instructions, when executed by the one or more processors, further cause the one or more processors to render for human perception and display the acquired ECG signal, or the pre-processed ECG signal, or the extracted QRS complexes, or the denoised QRS complexes, or some combination thereof.

In a thirteenth embodiment, a technological system for delivering care to a patient comprises a patient monitor, a network over which the patient monitor transmits signals; and a computing device receiving the signals transmitted by the patient monitor over the network. The patient monitor comprises a sensor interface through which the patient monitor may receive acquired electrocardiogram (“ECG”) signals; a communications interface; a display; one or more processors; and a memory encoded with instructions. The instructions, when executed by the one or more processors, cause the one or more processors to: perform a computer-implemented method for mitigating noise from QRS complexes extracted from an electrocardiogram (“ECG”) signal, transmit the acquired ECG signal, or the pre-processed ECG signal, or the extracted QRS complexes, or the denoised QRS complexes, or some combination thereof; and render for human perception and display the acquired ECG signal, or the pre-processed ECG signal, or the extracted QRS complexes, or the denoised QRS complexes, or some combination thereof. The computer-implemented method for mitigating noise from QRS complexes comprises: pre-processing an acquired ECG signal to obtain a pre-processed ECG signal; identifying narrow pulses in the pre-processed ECG signal; extracting QRS complexes from the acquired ECG signal; cross-checking the extracted QRS complexes with the identified narrow pulses; and rejecting pulses in the extracted QRS complexes that synchronize with the identified narrow pulses to obtain denoised QRS complexes. The pre-processing includes applying a first bandpass filter comprised of finite and infinite impulse response filters; and applying a second bandpass filter comprised of an infinite impulse response filter;

In a fourteenth embodiment, in the technological system of the thirteenth embodiment, identifying narrow pulses in the pre-processed ECG signal includes: taking the absolute value of the pre-processed ECG signal; convolving the absolute value of the pre-processed ECG signal with a triangular impulse response to obtain an intermediate signal; identifying candidate pulses in the intermediate signal with amplitudes exceeding a predetermined threshold; and outputting candidate pulses that do not repeat within a predetermined time window as narrow pulses.

In a fifteenth embodiment, in the technological system of the thirteenth embodiment, identifying narrow pulses in the pre-processed ECG signal is performed in parallel with extracting QRS complexes from the acquired ECG signal.

In a sixteenth embodiment, in the technological system of the thirteenth embodiment, pre-processing the acquired ECG signal further includes: decimating the acquired ECG signal by a predetermined factor; and applying a notch filter to the decimated ECG signal.

In a seventeenth embodiment, in the technological system of the thirteenth embodiment, extracting the QRS complexes from the pre-processed ECG signal includes: removing baseline wander to obtain an intermediate signal; decimating the intermediate signal by a predetermined factor; and detecting the QRS complexes in the decimated intermediate signal.

In an eighteenth embodiment, in the technological system of the thirteenth embodiment, the computing device is located at a centralized monitoring station displays the signals transmitted by the patient monitor for a caregiver to monitor.

In a nineteenth embodiment, in the technological system of the thirteenth embodiment, the computing device is located at a remote location and displays the signals transmitted by the patient monitor for a caregiver to monitor.

In a twentieth embodiment, the technological system of the thirteenth embodiment further comprises an electronic medical records repository including at least electronic medical record in which the e signals transmitted by the patient monitor are stored for later retrieval.

The detailed description is made with reference to the accompanying drawings and is provided to assist in a comprehensive understanding of various example embodiments of the present disclosure. Changes may be made in the function and arrangement of elements discussed without departing from the spirit and scope of the disclosure. Various embodiments may omit, substitute, or add various procedures or components as appropriate. For instance, features described with respect to certain embodiments may be combined in other embodiments. In addition, descriptions of well-known functions and constructions may be omitted for clarity and conciseness. Accordingly, those of ordinary skill in the art will recognize that various changes and modifications of the examples described herein can be made without departing from the spirit and scope of the present disclosure.

The expressions such as “include” and “may include” which may be used in the present disclosure denote the presence of the disclosed functions, operations, and constituent elements, and do not limit the presence of one or more additional functions, operations, and constituent elements. In the present disclosure, terms such as “include” and/or “have”, may be construed to denote a certain characteristic, number, operation, constituent element, component or a combination thereof, but should not be construed to exclude the existence of or a possibility of the addition of one or more other characteristics, numbers, operations, constituent elements, components or combinations thereof.

As used herein, the article “a” is intended to have its ordinary meaning in the patent arts, namely “one or more.” Herein, the term “about” when applied to a value generally means within the tolerance range of the equipment used to produce the value, or in some examples, means plus or minus 10%, or plus or minus 5%, or plus or minus 1%, unless otherwise expressly specified. Further, herein the term “substantially” as used herein means a majority, or almost all, or all, or an amount with a range of about 51% to about 100%, for example. Moreover, examples herein are intended to be illustrative only and are presented for discussion purposes and not by way of limitation.

As used herein, to “provide” an item means to have possession of and/or control over the item. This may include, for example, forming (or assembling) some or all of the item from its constituent materials and/or, obtaining possession of and/or control over an already-formed item.

Unless otherwise defined, all terms including technical and/or scientific terms used herein have the same meaning as commonly understood by one of ordinary skill in the art to which the present disclosure pertains. In addition, unless otherwise defined, all terms defined in generally used dictionaries may not be overly interpreted. In the following, details are set forth to provide a more thorough explanation of the embodiments. However, it will be apparent to those skilled in the art that embodiments may be practiced without these specific details. In other instances, well-known structures and devices are shown in block diagram form or in a schematic view rather than in detail in order to avoid obscuring the embodiments. In addition, features of the different embodiments described hereinafter may be combined with each other, unless specifically noted otherwise. For example, variations or modifications described with respect to one of the embodiments may also be applicable to other embodiments unless noted to the contrary.

Further, equivalent or like elements or elements with equivalent or like functionality are denoted in the following description with equivalent or like reference numerals. As the same or functionally equivalent elements are given the same reference numbers in the figures, a repeated description for elements provided with the same reference numbers may be omitted. Hence, descriptions provided for elements having the same or like reference numbers are mutually exchangeable.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

In the present disclosure, expressions including ordinal numbers, such as “first”, “second”, and/or the like, may modify various elements. However, such elements are not limited by the above expressions. For example, the above expressions do not limit the sequence and/or importance of the elements. The above expressions are used merely for the purpose of distinguishing an element from the other elements. For example, a first box and a second box indicate different boxes, although both are boxes. For further example, a first element could be termed a second element, and similarly, a second element could also be termed a first element without departing from the scope of the present disclosure.

A sensor refers to a component which converts a physical quantity to be measured to an electric signal, for example, a current signal or a voltage signal. The physical quantity may for example comprise electromagnetic radiation (e.g., photons of infrared or visible light), a magnetic field, an electric field, a pressure, a force, a temperature, a current, or a voltage, but is not limited thereto.

ECG signal processing, as used herein, refers to, without limitation manipulating an analog signal in such a way that the signal meets the requirements of a next stage for further processing. ECG signal processing may include converting between analog and digital realms (e.g., via an analog-to-digital or digital-to-analog converter), amplification, filtering, converting, biasing, range matching, isolation and any other processes required to make a sensor output suitable for processing.

Use of the phrases “capable of,” “capable to,” “operable to,” “configured to,” or “programmed to” in one or more embodiments, refers to some apparatus, logic, hardware, and/or element designed in such a way to enable the use of the apparatus, logic, hardware, and/or element in a specified manner. Use of the phrase “exceed” in one or more embodiments, indicates that a measured value could be higher than a pre-determined threshold (e.g., an upper threshold), or lower than a pre-determined threshold (e.g., a lower threshold). When a pre-determined threshold range (defined by an upper threshold and a lower threshold) is used, the use of the phrase “exceed” in one or more embodiments could also indicate a measured value is outside the pre-determined threshold range (e.g., higher than the upper threshold or lower than the lower threshold). The subject matter of the present disclosure is provided as examples of apparatus, systems, methods, circuits, and programs for performing the features described in the present disclosure. However, further features or variations are contemplated in addition to the features described above. It is contemplated that the implementation of the components and functions of the present disclosure can be done with any newly arising technology that may replace any of the above-implemented technologies.

Various modifications to the disclosure will be readily apparent to those skilled in the art, and the generic principles defined herein may be applied to other variations without departing from the spirit or scope of the present disclosure. Throughout the present disclosure the terms “example,” “examples,” or “exemplary” indicate examples or instances and do not imply or require any preference for the noted examples. Thus, the present disclosure is not to be limited to the examples and designs described herein but is to be accorded the widest scope consistent with the principles and novel features disclosed.

This concludes the detailed description. The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed:

1. A computer-implemented method for mitigating noise from QRS complexes extracted from an electrocardiogram (“ECG”) signal, comprising:
pre-processing an acquired ECG signal to obtain a pre-processed ECG signal, including:
applying a first bandpass filter comprised of finite and infinite impulse response filters; and
applying a second bandpass filter comprised of an infinite impulse response filter;
identifying narrow pulses in the pre-processed ECG signal;
extracting QRS complexes from the acquired ECG signal;
cross-checking the extracted QRS complexes with the identified narrow pulses; and
rejecting pulses in the extracted QRS complexes that synchronize with the identified narrow pulses to obtain denoised QRS complexes.

2. The computer-implemented method of claim 1, wherein identifying narrow pulses in the pre-processed ECG signal includes:
taking the absolute value of the pre-processed ECG signal;
convolving the absolute value of the pre-processed ECG signal with a triangular impulse response to obtain an intermediate signal;
identifying candidate pulses in the intermediate signal with amplitudes exceeding a predetermined threshold; and
outputting candidate pulses that do not repeat within a predetermined time window as narrow pulses.

3. The computer-implemented method of claim 1, wherein identifying narrow pulses in the pre-processed ECG signal is performed in parallel with extracting QRS complexes from the acquired ECG signal.

4. The computer-implemented method of claim 1, wherein pre-processing the acquired ECG signal further includes:
decimating the acquired ECG signal by a predetermined factor; and
applying a notch filter to the decimated ECG signal.

5. The computer-implemented method of claim 1, wherein extracting the QRS complexes from the pre-processed ECG signal includes:
removing baseline wander to obtain an intermediate signal;
decimating the intermediate signal by a predetermined factor; and
detecting the QRS complexes in the decimated intermediate signal.

6. A patient monitor, comprising:
a sensor interface through which the patient monitor may receive acquired electrocardiogram (“ECG”) signals;
one or more processors; and
a memory encoded with instructions that, when executed by the one or more processors, cause the one or more processors to perform a computer-implemented method for mitigating noise from QRS complexes extracted from an electrocardiogram (“ECG”) signal, the computer-implemented method comprising:
pre-processing an acquired ECG signal to obtain a pre-processed ECG signal, including:
applying a first bandpass filter comprised of finite and infinite impulse response filters; and
applying a second bandpass filter comprised of an infinite impulse response filter;
identifying narrow pulses in the pre-processed ECG signal;
extracting QRS complexes from the acquired ECG signal;
cross-checking the extracted QRS complexes with the identified narrow pulses; and
rejecting pulses in the extracted QRS complexes that synchronize with the identified narrow pulses to obtain denoised QRS complexes.

7. The patient monitor of claim 6, wherein identifying narrow pulses in the pre-processed ECG signal includes:
taking the absolute value of the pre-processed ECG signal;
convolving the absolute value of the pre-processed ECG signal with a triangular impulse response to obtain an intermediate signal;
identifying candidate pulses in the intermediate signal with amplitudes exceeding a predetermined threshold; and
outputting candidate pulses that do not repeat within a predetermined time window as narrow pulses.

8. The patient monitor of claim 6, wherein identifying narrow pulses in the pre-processed ECG signal is performed in parallel with extracting QRS complexes from the acquired ECG signal.

9. The patient monitor of claim 6, wherein pre-processing the acquired ECG signal further includes:
decimating the acquired ECG signal by a predetermined factor; and
applying a notch filter to the decimated ECG signal.

10. The patient monitor of claim 6, wherein extracting the QRS complexes from the pre-processed ECG signal includes:
removing baseline wander to obtain an intermediate signal;
decimating the intermediate signal by a predetermined factor; and
detecting the QRS complexes in the decimated intermediate signal.

11. The patient monitor of claim 6, further comprising:
a communications interface, and wherein:
the instructions, when executed by the one or more processors, further cause the one or more processors to transmit the acquired ECG signal, or the pre-processed ECG signal, or the extracted QRS complexes, or the denoised QRS complexes, or some combination thereof.

12. The patient monitor of claim 6, further comprising:
a display, and wherein:
the instructions, when executed by the one or more processors, further cause the one or more processors to render for human perception and display the acquired ECG signal, or the pre-processed ECG signal, or the extracted QRS complexes, or the denoised QRS complexes, or some combination thereof.

13. A technological system for delivering care to a patient, the technological system comprising:
a patient monitor, comprising:
a sensor interface through which the patient monitor may receive acquired electrocardiogram (“ECG”) signals;
a communications interface;
a display;
one or more processors; and
a memory encoded with instructions that, when executed by the one or more processors, cause the one or more processors to:
perform a computer-implemented method for mitigating noise from QRS complexes extracted from an electrocardiogram (“ECG”) signal, the computer-implemented method comprising:
pre-processing an acquired ECG signal to obtain a pre-processed ECG signal, including:
applying a first bandpass filter comprised of finite and infinite impulse response filters; and
applying a second bandpass filter comprised of an infinite impulse response filter;
identifying narrow pulses in the pre-processed ECG signal;
extracting QRS complexes from the acquired ECG signal;
cross-checking the extracted QRS complexes with the identified narrow pulses; and
rejecting pulses in the extracted QRS complexes that synchronize with the identified narrow pulses to obtain denoised QRS complexes;
transmit the acquired ECG signal, or the pre-processed ECG signal, or the extracted QRS complexes, or the denoised QRS complexes, or some combination thereof; and
render for human perception and display the acquired ECG signal, or the pre-processed ECG signal, or the extracted QRS complexes, or the denoised QRS complexes, or some combination thereof;
a network over which the patient monitor transmits signals; and
a computing device receiving the signals transmitted by the patient monitor over the network.

14. The technological system of claim 13, wherein identifying narrow pulses in the pre-processed ECG signal includes:
taking the absolute value of the pre-processed ECG signal;
convolving the absolute value of the pre-processed ECG signal with a triangular impulse response to obtain an intermediate signal;
identifying candidate pulses in the intermediate signal with amplitudes exceeding a predetermined threshold; and
outputting candidate pulses that do not repeat within a predetermined time window as narrow pulses.

15. The technological system of claim 13, wherein identifying narrow pulses in the pre-processed ECG signal is performed in parallel with extracting QRS complexes from the acquired ECG signal.

16. The technological system of claim 13, wherein pre-processing the acquired ECG signal further includes:
decimating the acquired ECG signal by a predetermined factor; and
applying a notch filter to the decimated ECG signal.

17. The technological system of claim 13, wherein extracting the QRS complexes from the pre-processed ECG signal includes:
removing baseline wander to obtain an intermediate signal;
decimating the intermediate signal by a predetermined factor; and
detecting the QRS complexes in the decimated intermediate signal.

18. The technological system of claim 13, wherein:
the computing device is located at a centralized monitoring station; and
the computing device displays the signals transmitted by the patient monitor for a caregiver to monitor.

19. The technological system of claim 13, wherein:
the computing device is located at a remote location; and
the computing device displays the signals transmitted by the patient monitor for a caregiver to monitor.

20. The technological system of claim 13, further comprising an electronic medical records repository including at least electronic medical record in which the e signals transmitted by the patient monitor are stored for later retrieval.
